SEQUENCE LISTING (1 sequence)

SEQ ID NO: 1 - protein, 535 aa, Artificial Sequence
  Modif. Diphteria toxin
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

(19) United States
(12) Patent Application Publication
(10) Pub. No.: US 2024/0228529 A9
(43) Pub. Date: Jul. 11, 2024

(54) METHOD FOR PURIFYING A PROTEIN OF INTEREST

(71) Applicant: XPRESS BIOLOGICS, Herstal (BE)

(72) Inventors: Marc DAUKANDT, Bilstain (BE); Philippe LEDENT, Liège (BE); Christian RODRIQUEZ, Spa (BE)

(73) Assignee: XPRESS BIOLOGICS, Herstal (BE)

(21) Appl. No.: 18/279,170

(22) PCT Filed: Feb. 28, 2022
(86) PCT No.: PCT/EP2022/054912

§ 371 Date: Aug. 28, 2023

(30) Foreign Application Priority Data
Feb. 26, 2021 (BE) BE2021/5138

(51) Int. Cl. C07K 1/18; C07K 1/36

(52) U.S. Cl. CPC C07K 1/18; C07K 1/36

(57) ABSTRACT

A method is for purifying a biomolecule (protein, DNA) of interest from a culture of Gram-negative bacteria uses polyoxyethylene isooctylcyclohexyl ether to remove endotoxins. Polyoxyethylene isooctylcyclohexyl ether is used to remove endotoxins and a solution having a pH between 8.0 and 9.0 and a concentration of salts from 2.5M to 3M in chromatography is used for specific leaching of bacterial contaminants.

### TECHNICAL FIELD

The present invention relates to purifying a protein of interest obtained by fermenting a Gram-negative bacterium, in particular to purifying endotoxins without using phenolic solvents.

### TECHNOLOGICAL BACKGROUND OF THE INVENTION

Fermenting proteins from Gram-negative bacteria is currently used for reasons of ease and cost.

However, these bacteria synthesise lipopolysaccharide-type components, also known as ‘endotoxins’, which are difficult to remove.

These components are particularly problematic when they are associated with proteins intended to be administered to a patient, in particular when large quantities must be administered, for example in therapeutic vaccination, since this means co-administering larger quantities of endotoxins to the patient.

### PRIOR ART

Various methods of chromatography and washing using phenolic detergents such as Triton® X-100 have been developed that enable a protein of interest synthesised by a Gram-negative bacterium to be purified.

However, these methods either do not sufficiently remove endotoxins, or involve using phenolic detergents, which means environmental problems, or involve or require a specific chromatography to be added, using a membrane such as Mustang® or Sartobind®, which is not very effective.

“Detergents for nucleic acid purification in anion exchange chromatography” Research Disclosure, 2021, ISSN 0474-4353 compares in static mode the properties of different detergents, including reduced Triton X-100, for purifying DNA containing endotoxins. After treatment, 0.4 endotoxin units (EU)/μg of plasmid remain, compared with less than 0.30 when washed with Triton X-100, which are high values; other detergents enable better purification.

Document US2003/041346 describes the use of reduced Triton X-100 in a method for purifying the enzyme P450ox involving partitioning with Triton X-114.

### SUMMARY OF THE INVENTION

A first aspect of the present invention is a method for purifying a biomolecule of interest containing endotoxins from a bacteria culture (e.g. Gram-negative, possibly Gram-positive) comprising the following steps:
conditioning the biomolecule of interest in a solution and
a chromatography step, wherein (i) the biomolecule of interest is bound to a support, (ii) the biomolecule of interest bound to the support is washed with a solution comprising polyoxyethylene (10) isooctylcyclohexyl ether, then wherein the biomolecule of interest bound to the support is washed with a solution free of polyoxyethylene (10) isooctylcyclohexyl ether and (iii) the biomolecule of interest bound to the support is eluted. Preferably, the biomolecule of interest is DNA, RNA or a protein.

Preferably, between 0.01% and 1% (w:v), preferably between 0.05 and 0.5% (w:v), favourably from 0.1 to 0.2% (w:v), such as about 0.15% (w:v) of polyoxyethylene (10) isooctylcyclohexyl ether is used during the washing step comprising this molecule.

Advantageously, the support for binding the biomolecule is a chromatography, preferably, this chromatography comprising a loading step, a washing step and an elution step.

Preferably, in this method, the chromatography uses an anion exchange resin.

Preferably, the endotoxins are removed using a concentration of polyoxyethylene (10) isooctylcyclohexyl ether between 0.01% and 1% (w:v), preferably between 0.05 and 0.5% (w:v), favourably from 0.1 to 0.2% (w:v), such as about 0.15% (w:v).

Preferably, the endotoxins of the chromatography are removed with a volume between 2 and 20 times the column volume, preferably a volume of about 5 times the column volume (CV).

Therefore, typically, at least 2 column volumes (CV), preferably at least 3 CV, preferably between 2 and 10 CV or between 3 and 7 CV, favourably about 5 CV of a solution comprising polyoxyethylene (10) isooctylcyclohexyl are applied to the chromatography, followed by at least 2 column volumes (CV), preferably at least 3 CV, of a washing solution free of detergent (polyoxyethylene (10) isooctylcyclohexyl), preferably between 2 and 10 CV or between 3 and 7 CV, favourably about 5 CV.

This enables the endotoxin content (EU) to be reduced to 20%, preferably equal to or less than 10%, more preferably equal to or less than 5% or even more preferably equal to or less than 2%, favourably equal to or less than 1% relative to an EU quantity before this step.

In practice, when the biomolecule of interest is a plasmid, it is loaded onto the column, for example an anion exchange resin, then equilibrated in a solution free of detergent and having a suitable ionic strength so that the plasmid is bound to the column, for example the ionic strength is advantageously between 10 and 60 mS/cm (the salt is advantageously substantially NaCl; the salt is added so as to ensure binding of the biomolecule of interest, but as little binding of contaminants as possible; minimum values of 20, 30, 40, even 50 mS/cm are preferred, in particular a value range between 40 and 60 mS/cm (even between 50 and 60 mS/cm) is particularly preferred, because it provides good binding capacity while simplifying washing and elution).

Typically, between 2 and 10 CV, for example between 3 and 7 CV of such solution are applied to the column. Next, the same solution enriched with detergent (polyoxyethylene (10) isooctylcyclohexyl) is applied to the column (as written above, typically between 2 and 10 CV, for example between 3 and 7 CV), before washing without detergent, then elution, for example by applying a much more concentrated saline solution, for example 1 mol/litre NaCl.

Advantageously, elution occurs by applying a gradient wherein the (or a) solution with a conductivity between 50 and 60 mS/cm is progressively replaced by the solution comprising 1 mol/litre NaCl. This ensures reproducible elution and quick release of the plasmid. Such a gradient may be linear or non-linear.

A linked aspect of the present invention is a method for purifying a protein of interest from a bacteria culture (e.g. Gram-negative, possibly Gram-positive) which comprises the protein of interest, the method comprising the following steps: (a) conditioning the protein of interest in a solution, (b) a series of purification steps to recover the protein of interest in a first fraction to be retained and contaminants in one or more fractions to be removed, said contaminants comprising endotoxins, wherein removing endotoxins comprises a step of adding a solution comprising polyoxyethylene (10) isooctylcyclohexyl ether.

Preferably, the series of purification steps of this method comprises a capture chromatography using an anion exchange resin and/or a hydrophobic interaction chromatography, advantageously, a first capture chromatography using an anion exchange resin and then a second hydrophobic interaction chromatography are performed, these first chromatography and second chromatography each comprising a loading step, a washing step and an elution step.

Advantageously, endotoxins are removed during the washing step of the first chromatography and/or the second chromatography, each chromatography advantageously comprising a loading step, a washing step and an elution step.

Preferably, the endotoxins are removed using a concentration of polyoxyethylene (10) isooctylcyclohexyl ether between 0.01% and 1% (w:v), preferably between 0.05 and 0.5% (w:v), favourably from 0.1 to 0.2% (w:v), such as about 0.15% (w:v).

Preferably, endotoxins of the first chromatography and/or second chromatography are removed with a volume between 1 and 10 times the column volume, preferably a volume of about 5 times the column volume.

This method is particularly advantageous when the protein of interest is SEQ ID NO: 1 and/or when the protein of interest is secreted in the periplasmic space of a Gram-negative bacterium.

Preferably, in this method, the capture chromatography using an anion exchange resin has a strong exchanger and/or the hydrophobic interaction chromatography is of the phenyl type.

Preferably, the loading and/or washing step of chromatography using a hydrophobic support is carried out at a pH between 8 and 9 and/or in the presence of a concentration of salts between 2 and 3M and/or has a load ratio between 0.1 g/L and 5 g/L, preferably between 0.3 and 2 g/L.

Preferably, the elution step of hydrophobic interaction chromatography is carried out with a solution with an electrical conductivity less than 105 mS/cm, preferably less than 80 mS/cm and/or more than 40 mS/cm.

Preferably, the loading step of the anion exchange chromatography is carried out at a load ratio between 2.5 and 3.5 g/L (protein weight: volume) and/or the elution step is carried out using a solution with an electrical conductivity greater than or double that used during the loading and/or washing step.

Preferably, this purification further comprises a step of passing through a Mustang® Q, Mustang® E or Sartobind® Q or Sartobind® STIC anion exchange membrane and/or a sterile filtration step.

Another linked aspect of the present invention is the use of polyoxyethylene (10) isooctylcyclohexyl ether to remove endotoxins in a composition, preferably further comprising a biomolecule of interest selected from DNA (plasmid), (m)RNA and a peptide, preferably a peptide.

This is advantageous for pharmaceutical use of this composition comprising a biomolecule of interest, preferably for vaccination, such as therapeutic vaccination.

Another linked aspect of the present invention is the use of a washing solution having a pH between 8.0 and 9.0 and a concentration of salts from 2.5M to 3M for specific leaching of bacterial contaminants from chromatography columns using a hydrophobic support using a phenyl group.

Preferably, this washing solution further comprises polyoxyethylene (10) isooctylcyclohexyl ether.

Another linked aspect of the present invention is the use of a solution having a pH between 7.5 and 8.5, preferably 8.0±0.2 and a conductivity of 12.0 mS/cm or less, preferably less than 10 mS/cm, and/or greater than 7 mS/cm to separate a biomolecule of interest from the bacterial contaminant LivK using anion exchange chromatography, preferably strong anion exchange chromatography.

### DESCRIPTION OF THE INVENTION

Numerous biomolecules of interest, for example peptides, are produced by fermenting Gram-negative bacteria in which, for example, the peptide of interest is grafted to a targeting sequence to the periplasmic space, simplifying recovery of the mature peptide. One of the inherent problems with this approach or other approaches to fermentation in microorganisms is the presence of endotoxins (EU), which means they have to be removed, at least up to an acceptable threshold. Using phenolic detergents like ‘Triton®’ reduces the EU content. However, this approach poses environmental problems. The inventors identified that polyoxyethylene (10) isooctylcyclohexyl ether could advantageously be used, although it is based on an unsaturated central structure, unlike the phenol (aromatic) group of Triton® X-100, whereas other non-phenolic detergents do not remove EUs effectively or cause downstream problems.

The present invention therefore relates to a method for purifying a biomolecule (protein) of interest from a Gram-negative bacteria culture. This method comprises removing EUs by washing with polyoxyethylene (10) isooctylcyclohexyl ether. One particularly advantageous protein is CRM 197 (SEQ ID NO: 1). This protein is, preferably, secreted in the periplasmic space of Escherichia coli. However, the present invention applies to SEQ ID NO: 1 obtained by other means (e.g. not targeted in the periplasmic space, cytosolic, secreted, fermented by other Gram-negative host cells, even in other microorganisms that would also produce endotoxins), or to other proteins, even to other biomolecules produced in such bacteria that could be injected into a patient, for example DNA (e.g. plasmid) or RNA (e.g. messenger RNA).

The protein of interest, when it is expressed in the periplasmic space, may advantageously be recovered by causing an osmotic shock, which releases the periplasmic contents and thus enables a large quantity of contaminating bacterial products, still trapped in the cell, to be easily separated.

Preferably, the present method comprises a preliminary step of recovering the protein of interest (e.g. SEQ ID NO: 1) in a buffer solution.

Advantageously, this purification method comprises a series of chromatography steps, preferably, a capture chromatography using an anion exchange resin and/or a chromatography using a hydrophobic support (hydrophobic interaction chromatography).

Preferably, each chromatography step comprises (each comprising) a loading step, a washing step and an elution step.

This makes it possible to combine washing in the presence of polyoxyethylene (10) isooctylcyclohexyl ether with one or both chromatography washing step(s), the capture chromatography using an anion exchange resin and/or the chromatography using a hydrophobic support (hydrophobic interaction chromatography).

Thus, when the protein of interest is loaded onto the chromatography column, the washing step with a solution comprising polyoxyethylene (10) isooctylcyclohexyl ether enables the majority, even almost all, of the EUs to be leached, without adding a specific step.

Preferably, the series of chromatography steps are carried out sequentially, a first capture chromatography using an anion exchange resin and then a second chromatography using a hydrophobic support (hydrophobic interaction chromatography).

Advantageously, the polyoxyethylene (10) isooctylcyclohexyl ether content (used during the washing step of one or both chromatography step(s)) is between 0.01% and 0.75% (w:v), preferably between 0.05 and 0.3% (w:v), favourably from 0.1 to 0.2% (w:v), such as about 0.15% (w:v).

Preferably, no phenolic solvent is used throughout the method, in particular during washing and/or EU removal steps.

Preferably, this removal of endotoxins is carried out with a volume of washing solution (comprising the polyoxyethylene (10) isooctylcyclohexyl ether) between 1 and 20 times the column volume (CV), preferably between 2 and 10 CV, or between 3 and 7 CV, favourably about 5 CV.

Advantageously, this endotoxin removal step is followed by a washing step with the same solution, but without the polyoxyethylene (10) isooctylcyclohexyl ether; volumes from 1 to 10 CV, for example about 5 CV, are preferred.

The inventors selected such high values, despite consequences in terms of (i) cost and (ii) washing time, to combine the application of the solution comprising polyoxyethylene (10) isooctylcyclohexyl ether, then of a solution which is free thereof, so that the protein of interest (SEQ ID NO: 1) is eluted in a solution free of this detergent.

Since polyoxyethylene (10) isooctylcyclohexyl ether does not have the specific absorbance property of Triton X 100 at 280 nm, the calculation of the washing volume required with the detergent-free solution can be done in a pre-test where polyoxyethylene (10) isooctylcyclohexyl ether is replaced by Triton X-100, and all other parameters are constant. Monitoring absorbance at 280 nm shows the desorption of Triton X-100, which gives an indication of the volume required for the desorption of polyoxyethylene (10) isooctylcyclohexyl ether. Since the pre-test is not intended to be repeated, it does not represent a major economic problem.

This enables the EU content to be reduced to 20%, preferably equal to or less than 10%, more preferably equal to or less than 5% or even more preferably equal to or less than 2%, favourably equal to or less than 1% relative to an EU quantity before this step.

This method also enables the protein of interest to be obtained with a purity equal to or greater than 65% (weight of the protein of interest: total protein weight), preferably equal to or greater than 70%, preferably equal to or greater than 73%, favourably equal to or greater than 75%.

This method also enables the protein of interest to be obtained in a composition having a residual amount of genomic DNA equal to or less than 5 ng/mg (genomic DNA weight: protein weight, i.e. the purified protein of interest), preferably equal to or less than 2 ng/mg, favourably equal to or less than 1 ng/mg.

Several types of anion exchange resins may be used in the present method, such as Q (quaternary amine) and DEAE (diethylaminoethyl) functions carried on a resin. For example, a strong “Q” exchange resin (in positive mode) was successfully used, particularly if the protein to be purified is that of SEQ ID NO: 1.

The anion exchange resin may have a bead size between 20 and 150 μm, preferably between 40 and 120 μm, preferably between 60 and 110 μm. The nature of the resin is selected and adapted according to the proteins to be purified.

The loading step of the capture chromatography using an anion exchange resin may be carried out at a pH between 5.0 and 11.0, preferably between 7.0 and 10.0, favourably between 8.0 and 9.0. When the protein to be purified is that of SEQ ID NO: 1, the loading step is preferably carried out at a pH between 7.0 and 10, such as from 8.0 to 9.0.

The solution used for the loading step preferably has an electrical conductivity between 2.0 and 15.0 mS (milli-Siemens)/cm, preferably between 5.0 and 10.0 mS/cm. The electrical conductivity of the solution is easily measured and, if required, adjusted by adding a salt, such as sodium chloride, or by diluting with demineralised water.

Preferably, the loading step is carried out using a composition comprising a load ratio of between 1 and 8 g/L, preferably between 2 and 5 g/L, favourably between 3 and 4 g/L. Good results were obtained with a load of 3.3 g/L, in particular, such a load made it possible to limit the retention of the contaminating protein LivK.

The washing step (see above) is, preferably, carried out with a solution having the same electrical conductivity as that of the loading step and the same pH; therefore, preferably, an electrical conductivity between 2.0 and 15.0 mS (milli-Siemens)/cm, preferably between 5.0 and 10.0 mS/cm.

The elution step is, preferably, carried out with a solution having an electrical conductivity greater than two times that of the loading step and/or between 9.0 and 20.0 mS/cm, preferably between 12.0 and 17.0 mS/cm.

The chromatography using a hydrophobic support (hydrophobic interaction chromatography) may be carried out using different resins, derivatised with hydrophobic groups selected from phenyl, butyl and hexyl. Good results were obtained using phenyl 650M resin. The pH for the loading is, preferably, between 5.0 and 11.0, preferably between 7.0 and 10.0, favourably between 8.0 and 9.0. In the case of SEQ ID NO: 1, the resin is preferably a phenyl resin, such as 650M phenyl resin, and the pH is preferably between 7 and 10, preferably between 8.0 and 9.0.

Preferably, the loading step using a hydrophobic resin (hydrophobic interaction chromatography) is carried out using a concentration of salts (e.g. sodium chloride) between 0.5 and 6M, preferably between 2 and 3M. In particular, when the protein to be purified is SEQ ID NO: 1, and the hydrophobic resin is of the phenyl type (650M phenyl), good results were obtained at high salt concentrations, such as from 2.0 to 3.0M.

Preferably, the load ratio of the hydrophobic resin is between 0.1 g/L and 5 g/L, preferably between 0.3 and 2 g/L.

When the protein to be purified is SEQ ID NO: 1 and/or a protein expressed in the periplasmic space, the washing step of the hydrophobic chromatography is advantageously carried out using a large volume (e.g. from 5 to 20 CV (column volume), such as about 15 CV) at a high pH (between 8.0 and 9.0, for example 8.5) and at a high salt concentration (between 2.3 and 3 M NaCl, for example about 2.6M NaCl). This enables the protein of the periplasmic space LivK to be leached, but not SEQ ID NO: 1 or another protein of interest expressed in the periplasmic space. In addition, like for the anion exchange chromatography, prolonged washing, despite its cost, makes it possible to insert a sub-step comprising polyoxyethylene (10) isooctylcyclohexyl ether, followed by a sub-step which is free thereof, so that the protein of interest is eluted in a solution free of this detergent.

After the chromatography steps, or even between the two chromatography steps, the solution may be filtered through a membrane. “Mustang E” membranes (cartridges) may be used after the hydrophobic interaction chromatography, which (further) reduces the endotoxin content. However, the inventors noted that this step was not usually necessary, since the washing during one or both chromatographies enabled compositions to be obtained with an EU content that is compatible with clinical use.

Lastly, advantageously, a (final) sterile filtration step using a filter with a porosity of 0.22 μm is carried out, so as to obtain a composition that can be injected into a patient.

A linked aspect of the present invention is a composition that can be injected into a mammal, in particular into a human (or into a dog, or into a horse or into a rabbit or into a camelid or into a monkey), comprising a protein of interest obtained by implementing the above purification method.

It is to be understood that the present invention is in no way limited to the embodiments described above and that modifications may be made without departing from the scope of the appended claims.

### Examples

### Example 1 Purifying a Protein of Interest

The protein of SEQ ID NO: 1 was produced by fermentation using a strain of Escherichia coli, under the control of a targeting peptide in the periplasmic space, under controlled conditions, for example as described in patent application BE2021/5137 filed on 26 Feb. 2021, so as to maximise the quantity of SEQ ID NO: 1 in the periplasmic space.

The extraction method selected to extract the SEQ ID NO: 1 involved an osmotic shock, which enabled the proteins to be extracted from the periplasmic compartment with a high yield, while minimising the release of membrane or cytoplasmic contaminants (proteins including certain proteases, endotoxins and residual DNA).

The extraction conditions successively involved a first re-suspension of the cells in a hypertonic buffer followed by dilution in a hypotonic solution. The experimental conditions (nature and ratio of the hyper- and hypotonic buffers) were tested on a small scale in batch mode, then transposed to a continuous tubular reactor.

The solubility of the proteins extracted from the periplasmic compartment and more particularly of SEQ ID NO: 1, was tested in increasing saline concentrations to check that there was no precipitation under conditions generally seen in liquid chromatography and/or to define the operational limits. It was shown that SEQ ID NO: 1 did not show any sign of precipitation in NaCl ranges from 0 to 3M, at pH 8.5. Because of this step, the inventors successfully obtained SEQ ID NO: 1 with a purity of already about 30%; SDS PAGE with successive 2 in 2 dilutions of the samples, staining of all the proteins.

Next, different chromatographic supports were tested. Among the anion exchangers, strong and weak exchangers (Q and DEAE functions), different types of matrix (agarose, methacrylate), structure (bead and sprawling structure), and physico-chemical conditions (pH and conductivity of the load fraction) were tested. Five matrices with increasing hydrophobicity (phenyl, butyl and hexyl functions) were also tested by comparing two salt concentrations for elution.

The resins Capto Q and Phenyl 650M were selected for their greater ability to bind SEQ ID NO: 1 than other resins and were then validated in dynamic mode. These tests were used to refine the chromatography parameters (pH, conductivity, quantity of protein loaded per volume of resin).

The purification sequence was developed to implement a two-step chromatography method to obtain a high yield and purity of the protein of interest greater than 95%. This research was carried out on the two types of resin selected during screening.
1. Capture by an anion exchange resin.
2. Polishing with a hydrophobic resin.

Different factors that may affect the purification of SEQ ID NO: 1 using an anion exchange resin were studied and compared with the screening results in order to establish an optimal chromatography sequence, i.e. a compromise between yield, purity and operation time. The scale-up was also tested in order to check that the method can be reproduced under conditions representative of industrial conditions.

The results obtained made it possible to optimise the capture step by selecting the best conditions:

Resin: Capto Q strong exchanger (better than DEAE), 90 μm beads, normal in terms of calibration. These choices maintained a high capacity for binding SEQ ID NO: 1 despite the lower degree of resolution of the chromatography compared to a Jetted calibrated resin. Four column heights were tested: 10, 12, 15 and 20 cm; the 12 cm height was preferred and four diameters (1.6; 2.6; 10 and 14 cm) were tested; the 14 cm diameter was preferred for the scale of purification considered.

Load: the physico-chemical conditions of the resin loading step were tested, i.e. a conductivity between 3.0 and 9.0 mS/cm and a pH of 8.5. These conditions enabled the protein to expose a high density of negative charges on its surface, required for binding to the resin (positive mode), while avoiding the binding of certain pigments and host proteins. The load ratio was also set at 3.3 g/L of resin (slight overload; 2.5 g/l and 4.2 g/l were also tested) and the flow rate at 90 cm/h, in order to create competition for binding sites between SEQ ID NO: 1 and the protein LivK (pI 5.2 vs 5.9), a periplasmic protein with an isoelectric point and hydrophobicity similar to the protein of interest. This competition limits the binding of this host protein.

Washing: the washing step was characterised by a volume of 10 CV or 15 CV (Column Volume; 5 and 20 CV were also tested) of buffer (this buffer will sometimes include 0.15% (w:v) polyoxyethylene (10) isooctylcyclohexyl ether; see below) at pH 8.5 and 7.0 mS/cm, at a flow rate of 300 cm/h. The purpose was to remove the majority of impurities not bound to the resin, including endotoxins, immediately after loading.

Elution: this step aimed to detach the protein of interest while minimising the co-elution of impurities bound to the resin. Since a proportion of the protein LivK was still on the resin and had a tendency to co-elute with SEQ ID NO: 1, elution therefore occurred in two steps. The first step consisted of a prolonged 15 CV wash at pH 8.0 with a solution of a similar conductivity to that of the solution of the washing step (e.g. 9 mS/cm), at a flow rate of 300 cm/h. The slight load difference of LivK enabled it to elute at a slightly lower conductivity than that used to elute SEQ ID NO: 1. The elution of SEQ ID NO: 1 itself was isocratic (gradient elution was also tested) and carried out at pH 8.5, with a much higher electrical conductivity than that of the washing solution (values from 9.0 to 20.0 mS/cm were tested), at 300 cm/h. Thus, the inventors noted that the eluted SEQ ID NO: 1 is cleared of proteins with high and low molecular weights (SDS PAGE analysis), as well as most pigments and endotoxins.

TABLE 1
comparison of the impact of different parameters used for the first
elution buffer applied before the CRM (SEQ ID NO: 1) elution
step by ion exchange chromatography using Capto Q.

| | | Conditions | | | | |
|---|---|---|---|---|---|---|
| | | pH | | | | |
| | Units | 8.5 | 8.5 | 8.5 | 8.0 | 7.5 |
| Conductivity | mS/cm | 9.0 | 10.5 | 12.0 | 9.0 | 9.0 |
| CRM load | g | 0.375 | 0.375 | 0.375 | 0.322 | 0.322 |
| CRM Elution | g | 0.263 | 0.191 | 0.094 | 0.229 | 0.133 |
| LivK Elution | g | 0.022 | 0.004 | 0 | 0.007 | 0.0016 |
| Rdt CRM | % | 70 | 51 | 25 | 71 | 41 |
| Ratio LivK/ | % | 8.4 | 2.1 | 0.0 | 3.1 | 1.2 |
| CRM | | | | | | |

NB. the LivK:CRM ratio is the ratio of the eluted fraction (see below).

Scale-up: the scale-up of the purification sequence was studied on different types of supports (HiScale and Axichrom columns) for different bed geometries (height of the resin bed, column diameter), but also with different packing techniques (Dynamic Axial Compression or DAC, incrementing the consolidation rate). As written above, the resin height was determined at 12 cm for a diameter of 14 cm, i.e. a resin volume of 1.846 L, to treat the equivalent of producing 5 L of culture from a bioreactor. The packing method selected consisted of incrementing the flow rate during the consolidation step by using water as the mobile phase. This protocol made it possible to obtain performance criteria (resolution and asymmetry) that meet the manufacturer's criteria for this resin. The electrical conductivity of the different solutions is easy to measure and is adapted by adding NaCl or by dilution. The electrical conductivity was precisely set for all solutions used. Because of this step, the inventors successfully increased the purity of SEQ ID NO: 1 to 80%; SDS PAGE with successive 2 in 2 dilutions of the samples, staining of all the proteins. A yield of 71% was obtained under the best conditions.

Developing a Polishing Step Using a Phenyl 650M Hydrophobic Support

Different factors that may affect the purification of SEQ ID NO: 1 using hydrophobic supports were studied and compared with the screening results in order to establish an optimal chromatography sequence, i.e. a compromise between yield, purity and operation time. The scale-up was also tested in order to check that the method can be reproduced under conditions representative of industrial conditions.

Different chromatography parameters were tested and optimised. Three bed heights (10 cm, 18.5 cm and 20 cm) as well as four diameters (from 1.6 cm to 14 cm) were tested. The values of 18.5 cm in height and 14 cm in diameter were preferred for the scale of production considered.

Resin: hydrophobic resin, 65 μm uncalibrated beads. This resin was selected during screening and placed in the polishing step in view of its reduced binding capacity compared to Capto Q.

Loading: the physico-chemical conditions of the resin loading step were a pH of 8.5 and a 2.6 M concentration of NaCl; values of 2.2, 2.4, 2.8 and 3M were tested. These conditions enabled the protein to expose the hydrophobic groups required for binding to the resin (positive mode), while avoiding the binding of certain pigments and host proteins.

Different ratios were tested, such as 0.5 g/L and 1.5 g/L. The load ratio was set to 0.8 g/L (very slight overload) in order to maintain a high purification yield. Several flow rates were tested, such as 60 cm/h and 150 cm/h.

Washing: the washing step consisted of a prolonged 15 CV wash at pH 8.5 and a 2.6M concentration of NaCl in order to destabilise the binding of LivK while minimising the detachment of SEQ ID NO: 1; this buffer will sometimes include 0.15% (w:v) polyoxyethylene (10) isooctylcyclohexyl ether; see below. The same NaCl concentrations and flow rates as for the load were tested. Four wash volumes were tested: 5 CV, 10 CV, 15 CV and 20 CV. 15 CV offered the best result.

Elution: this step aimed to detach the protein of interest while minimising the co-elution of impurities bound to the resin. Among the different conductivities tested, an isocratic elution at pH 8.5 with a high conductivity jump was selected because it enabled elution of a small volume (1 CV), which had a positive impact on the purity of SEQ ID NO: 1 but also on the duration of the chromatography and the following ultrafiltration step. In practice, numerous elution solutions were tested, from 155 mS/cm to less than 60 mS/cm.

Because of this step, the inventors successfully increased the purity of SEQ ID NO: 1 to 98%; SDS PAGE with successive 2 in 2 dilutions of the samples, staining of all the proteins. A 77% recovery yield of SEQ ID NO: 1 was obtained under the best conditions.

Scale-up: the scale-up of the purification sequence was studied on different types of supports (HiScale and Axichrom columns) for different bed geometries (resin bed height, column diameter) but also with different packing techniques (Dynamic Axial Compression, incrementing the consolidation flow rate). The resin height was determined at 18.5 cm for a diameter of 14 cm, i.e. a resin volume of 2.846 L, to treat the equivalent of producing 5 L of culture from a bioreactor in two sub-batches. The packing method selected consisted of using a very high constant flow rate (˜450 cm/h) during the consolidation step by using water as the mobile phase. This protocol made it possible to obtain performance criteria (resolution and asymmetry) that meet the manufacturer's criteria for this resin. However, scaling up did not enable an operational flow rate of 150 cm/h to be retained: this was lowered to 60 cm/h to avoid collapse of the resin bed when solutions with high salt concentrations were passed through it.

In order to formulate the protein, ultrafiltration membranes with different porosities (30, 50 and 70 kDa) were tested.

The first step of the ultrafiltration method consisted of concentrating the protein of interest to approximately 4.5 mg/mL and the second step consisted of diafiltering the concentrated retentate with a 10-fold larger volume of formulation buffer. The 30 kDa hollow fibre was selected for its better retention of the protein of interest (SEQ ID NO: 1).

The operational flow rate set to 20 L/m 2/h was applied throughout the method because it limits the shear factor during the method. A hollow fibre of 0.16 m² formulated a protein quantity equivalent to 5 L of culture in a bioreactor over a duration of 3 to 4 hours.

Removing endotoxins (during the washing step of the chromatography columns; already mentioned above).

The results demonstrated good efficiency in removing endotoxins for at least 3 conditions tested:

Capto Q; implementing a washing step with 0.15% v/v polyoxyethylene (10) isooctylcyclohexyl ether after loading and divided into 3 successive steps:

5 CV buffer (pH and conductivity, see above; e.g. between 3.0 and 9.0 mS/cm). The purpose of this step was to remove the majority of impurities not bound to the resin immediately after loading.

5 CV of the same buffer+1.5% polyoxyethylene (10) isooctylcyclohexyl ether. Here, polyoxyethylene (10) isooctylcyclohexyl ether removed the endotoxins by reducing them very significantly (<1 EU/mg in the elution fraction of SEQ ID NO: 1 compared to >1000 EU/mg without washing with polyoxyethylene (10) isooctylcyclohexyl ether), i.e. a reduction of 3 Log or even more compared to the same step without this detergent. Furthermore, using this detergent did not interfere with the binding of the protein of interest to the resin.

5 CV of the same buffer, but without polyoxyethylene (10) isooctylcyclohexyl ether. This step removed polyoxyethylene (10) isooctylcyclohexyl ether. In practice, removal of polyoxyethylene (10) isooctylcyclohexyl ether was evaluated in a pre-test of absorbance monitoring, all the conditions were the same, except that Triton X-100 was present instead of polyoxyethylene (10) isooctylcyclohexyl ether, which enabled monitoring at 280 nm.

Phenyl 650M; similarly to Capto Q:

5 CV of buffer at a high conductivity, e.g. at 150-200 mS/cm and pH 8.5. The purpose of this step was to remove the majority of impurities not bound to the resin immediately after loading.

5 CV of buffer at 150-200 mS/cm and pH 8.5+1.5% polyoxyethylene (10) isooctylcyclohexyl ether. Here, polyoxyethylene (10) isooctylcyclohexyl ether removed the endotoxins by reducing them very significantly (<1 EU/mg in the elution fraction of SEQ ID NO: 1 compared to >1000 EU/mg without washing with polyoxyethylene (10) isooctylcyclohexyl ether). Furthermore, using this detergent did not interfere with the binding of the protein of interest to the resin.

5 CV of buffer at 150-200 mS/cm and pH 8.5. This step removed polyoxyethylene (10) isooctylcyclohexyl ether (pre-test with absorbance monitoring at 280 nm; see above).

Other detergents were tested, both for ion exchange chromatography and hydrophobic interaction chromatography, such as Brij 35, Tween 20 or Tween 80 (ranges tested: 0.016 to 3%; w:v). However, these detergents were either less effective at removing endotoxins, or were incompatible with large-scale use.

The inventors also attempted an alternative method of removing endotoxins by filtering through a Mustang E matrix: this type of matrix contains, in its coating, quaternary ammonium groups with a high affinity for endotoxins. SEQ ID NO: 1 had little or even no affinity with this type of support under the conditions tested (pH, conductivity, flow rate). This step showed the same endotoxin removal efficiency when it was placed after ultrafiltration formulation or after purification using Capto Q (16 EU/mg after filtration) without significantly affecting the purification yield. A 10 ml filter treated a quantity of the purified product equivalent to 5 L of culture in a bioreactor. However, this alternative was not as effective as using polyoxyethylene (10) isooctylcyclohexyl ether but may be used as a complement.

### Example 2. Purifying a Plasmid of Interest

E. coli cells containing a plasmid of interest were cultivated to a high density in fed-batch mode in a SOL fermenter, to synthesise more than 250 mg of the plasmid of interest.

The cell suspension was subjected to alkaline lysis, followed by neutralisation and precipitation.

The mixture was then subjected to a series of filtrations to remove large debris, and then ultrafiltration to remove contaminants with low molecular weights.

The retentate of the latter filtration was bound to a TMAE resin equilibrated with a solution A comprising NaCl (pH here set to about 8.5 by a TRIS buffer; other pH values are possible as long as the resin remains positively charged and the DNA remains negatively charged), so as to have a conductivity between 50 and 60 mS/cm (the inventors noted that lower values are possible). Next, 5 times the column volume of the same solution A were applied to the column, then 5 times the column volume of the same solution A, further comprising 0.15% (by weight) polyoxyethylene (10) isooctylcyclohexyl ether were applied, and lastly 5 times the column volume of the same solution A free of polyoxyethylene (10) isooctylcyclohexyl ether were applied, before eluting with a gradient of a solution B (1 mol/l NaCl, pH 8.5): 10 CV linear gradient starting from 100% of solution A above having a conductivity between 50 and 60 mS/cm and ending with 100% of solution B above, then 2 CV of solution B only. Elution may be carried out differently, for example using non-linear gradients.

The fractions comprising the plasmid were then subjected to filtration steps.

According to the experiments, the start endotoxin content varied from 350 to 460 EU/mg of plasmid and was easily reduced to values of 5 EU/mg of plasmid and, in the best cases, to values of less than 1 EU/mg of plasmid.

1. A method for purifying a biomolecule of interest containing endotoxins from a bacteria culture comprising the following steps:
conditioning said biomolecule of interest in a solution, and
a chromatography step wherein
i. said biomolecule of interest is bound to a support,
ii. said biomolecule of interest bound to the support is washed with a solution comprising polyoxyethylene isooctylcyclohexyl ether, then said biomolecule of interest bound to the support is washed with a solution free of polyoxyethylene isooctylcyclohexyl ether, and
iii. said biomolecule of interest bound to the support is eluted, wherein the biomolecule of interest is DNA, and wherein at least 2 column volumes of the washing solution comprising polyoxyethylene isooctylcyclohexyl ether are applied to said support and at least 2 column volumes of the washing solution with a solution free of polyoxyethylene isooctylcyclohexyl ether are applied before elution, said washing solution having a conductivity between 10 and 60 mS/cm.
iv. preferably between 40 mS/cm and 60 mS/cm,
v. even more preferably between 50 and 60 mS/cm.

2. (canceled)

3. A method for purifying a protein of interest from a bacteria culture which comprises said protein of interest, said method comprising the following steps:
(a) conditioning said protein of interest in a solution,
(b) a series of purification steps to recover said protein of interest in a first fraction to be retained and contaminants in one or more fractions to be removed, said contaminants comprising endotoxins,
wherein removing endotoxins comprises a step of adding a solution comprising polyoxyethylene isooctylcyclohexyl ether, wherein the series of purification steps comprises capture chromatography using an anion exchange resin and/or hydrophobic interaction chromatography.

4. (canceled)

5. The method for purifying a protein of interest according to claim 3, wherein a first capture chromatography using an anion exchange resin and then a second hydrophobic interaction chromatography are performed, said first chromatography and second chromatography each comprising a loading step, a washing step and an elution step.

6. The method for purifying a protein of interest according to claim 3, wherein removing endotoxins is carried out during the washing step of the first chromatography and/or the second chromatography.

7. The method for purifying a protein of interest according to claim 3, wherein the endotoxins are removed using a concentration of polyoxyethylene isooctylcyclohexyl ether between 0.01% and 1% (w:v).

8. The method for purifying a protein of interest according to claim 3, wherein endotoxins of the first chromatography and/or second chromatography are removed with a volume between 1 and 10 times the column volume.

9. The method for purifying a protein of interest according to claim 3, wherein the protein of interest is SEQ ID NO: 1.

10. The method-for purifying a protein of interest according to claim 3, wherein the protein of interest is secreted into the periplasmic space of a Gram-negative bacterium.

11. The method for purifying a protein of interest according to claim 3, wherein the capture chromatography using an anion exchange resin has a strong exchanger.

12. The method for purifying a protein of interest according to claim 5, wherein the hydrophobic interaction chromatography is of the phenyl type.

13. The method for purifying a protein of interest according to claim 12, wherein the loading and/or washing step of the chromatography using a hydrophobic support is carried out at a pH between 8 and 9 and/or in the presence of a concentration of salts between 2 and 3M and/or has a load ratio between 0.1 g/L and 5 g/L.

14. The method for purifying a protein of interest according to claim 12, wherein the elution step of the hydrophobic interaction chromatography is carried out with a solution with an electrical conductivity of less than 105 mS/cm.

15. The method for purifying a protein of interest according to claim 5, wherein the loading step of the anion exchange chromatography is carried out at a load ratio between 2.5 and 3.5 g/L (protein weight: volume) and/or wherein the elution step is carried out using a solution with an electrical conductivity greater than or double the electrical conductivity used during the loading and/or washing step.

16. The method for purifying a protein of interest according to claim 3, further comprising a step of passing through a Mustang® Q, Mustang® E or Sartobind® Q or Sartobind® STIC anion exchange membrane.

17. The method according to claim 3, further comprising a sterile filtration step.

18-21. (canceled)

22. Use of a washing solution having a pH between 8.0 and 9.0 and a concentration of salts from 2.5M to 3M for specific leaching of bacterial contaminants from chromatography columns on a hydrophobic support with a phenyl group.

23-24. (canceled)

25. Use according to claim 22, wherein the washing solution further comprises polyoxyethylene isooctylcyclohexyl ether.
